(19) United States
(12) Patent Application Publication
(10) Pub. No.: US 2007/0123584 A1
(43) Pub. Date: May 31, 2007

(54) METHOD OF TREATING PREMENSTRUAL DYSPHORIC DISORDER WITH ESCITALOPRAM

(75) Inventor: Connie Sanchez, West Milford, NJ (US)

Correspondence Address: DARBY & DARBY P.C., P. O. BOX 5257, NEW YORK, NY, 10150-5257, US

(73) Assignee: H. Lundbeck A/S, Valby-Copenhagen (DK)

(21) Appl. No.: 11/625,554

(22) Filed: Jan. 22, 2007

Related U.S. Application Data
(63) Continuation of application No. 10/984,536, filed on Nov. 8, 2004.
(60) Provisional application No. 60/518,276, filed on Nov. 7, 2003.

(51) Int. Cl. A61K 31/343

(52) U.S. Cl. 514/469

(57) ABSTRACT

The present invention relates to a method of treating the symptoms of premenstrual dysphoric disorder (PMDD) in a patient in need thereof by administering an effective amount of escitalopram or a pharmaceutically acceptable salt thereof.

This application claims the benefit of U.S. Provisional Application No. 60/518,276, filed Nov. 7, 2003, which is hereby incorporated by reference.

### FIELD OF THE INVENTION

The present invention relates to a method of treating the symptoms of premenstrual dysphoric disorder (PMDD) in a patient in need thereof by administering an effective amount of escitalopram or a pharmaceutically acceptable salt thereof.

### BACKGROUND OF THE INVENTION

Escitalopram is an example of a class of drugs known as selective serotonin reuptake inhibitors (hereafter referred to as SSRIs). SSRIs are selective for the 5-HT-uptake of serotonin, and have been used for the treatment of depression. See, for example, U.S. Pat. No. Re. 34,712, which is hereby incorporated by reference.

Escitalopram is the S-enantiomer of citalopram arid has the following 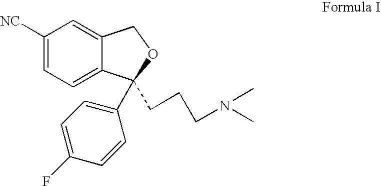
structure:

International Publication No. WO 02/087566-A1, which is hereby incorporated by reference, discloses the use of escitalopram to treat depression (such as major depression disorder), neurotic disorders, acute stress disorder, eating disorders (e.g., bulimia, anorexia, and obesity), phobias, dysthymi, pre-menstrual syndrome, cognitive disorders, impulse control disorders, attention deficit hyperactivity disorder, and drug abuse.

Women diagnosed with premenstrual dysphoric disorder (PMDD) have monthly symptoms which markedly interfere with work, school, usual social activities, and their relationships with others. The essential symptoms of PMDD according to the Diagnostic & Statistical Manual of Mental Diseases IV-TR (DSM IV-TR) include markedly depressed mood, marked anxiety, affective lability, and decreased interest in activities.

Wikander et al. report in the Journal of Clinical Psychopharmacology (1998, 18(5):390-398) that citalopram administered to women according to certain regiments in a clinical trial was found to be an effective treatment for PMDD.

There exists a continuing need for effective methods of treating the symptoms of premenstrual dysphoric disorder which have few, if any, side effects.

### SUMMARY OF THE INVENTION

The present invention relates to a method of treating a patient (or woman) suffering from premenstrual dysphoric disorder (PMDD) by administering an effective amount of escitalopram or a pharmaceutically acceptable salt thereof. The daily dose of escitalopram or a pharmaceutically acceptable salt thereof administered preferably ranges from about 5 to about 20 mg (calculated on a weight basis of escitalopram base). The daily does may be, for example, from about 5 to about 10 mg, from about 10 to about 20 mg, from about 5 mg, about 10 mg, or about 20 mg (calculated on a weight basis of escitalopram base). According to one preferred embodiment, the escitalopram or salt thereof is administered as a solid. The escitalopram or pharmaceutically acceptable salt thereof may be administered continuously throughout the menstrual cycle of the patient intermittently or semi-intermittently during the patient's menstrual cycle. The escitalopram or salt thereof is preferably administered orally (e.g., as a solid dosage form such as a tablet or capsule) although other routes of administration may be used. According to a preferred embodiment, it is administered during the morning.

### DETAILED DESCRIPTION OF THE INVENTION

The term escitalopram refers to (S)-(+)-1-[3-dimethylamino)propyl]-1-(4-fluorophenyl)-1,3-dihydro-5-isobenzofurancarbonitrile. Preferably, the escitalopram is at least 95, 96, 97, 98, 99, or 99.5% pure. More preferably, it contains less than 1 or 2% w/w of the corresponding R-enantiomer.

As used herein, the term “about” means within 10% of a given value, preferably within 5%, and more preferably within 1% of a given value. Alternatively, the term “about” means that a value can fall within a scientifically acceptable error range for that type of value, which will depend on how qualitative a measurement can be given the available tools.

As used herein, the term “premenstrual dysphoric disorder” or “PMDD” refers to a woman whose symptoms meet the criteria defined by DSM-IV-TR, which is hereby incorporated by reference. These criteria are reproduced below:
A. In most menstrual cycles during the past year, five (or more) of the following symptoms were present for most of the time during the last week of the menstrual phase, began to remit within a few days after the onset of the follicular phase, and were absent in the week postmenses, with at least one of the symptoms being either (1), (2), (3), or (4):
(1) markedly depressed mood, feelings of hopelessness, or self-deprecating thoughts
(2) marked anxiety, tension, feelings of being “keyed up,” or “on edge”
(3) marked affective lability (e.g., feeling suddenly sad or tearful or increased sensitivity to rejection)
(4) persistent and marked anger or irritability or increased interpersonal conflicts
(5) decreased interest in usual activities (e.g., work, school, friends, and hobbies)
(6) a subjective sense of difficulty in concentrating
(7) lethargy, easy fatigability, or marked lack of energy
(8) marked change in appetite, overeating, or specific food cravings
(9) hypersomnia or insomnia
(10) a subjective sense of being overwhelmed or out of control
(11) other physical symptoms, such as breast tenderness or swelling, headaches, joint or muscle pain, a sensation of “bloating,” weight gain
Note: In menstruating females, the luteal phase corresponds to the period between ovulation and the onset of menses, and the follicular phase begins with menses. In nonmenstruating females (e.g., those who have had a hysterectomy), the timing of menstrual and follicular phases may require measurement of circulating reproductive hormones.
B. The disturbance markedly interferes with work or school or with usual social activities and relationships with others (e.g., avoidance of social activities, decreased productivity and efficiency at work or school).
C. The disturbance is not merely an exacerbation of the symptoms of another disorder, such as Major Depressive Disorder, Panic Disorder, Dysthymic Disorder, or a Personality Disorder (although it may be superimposed on any of these disorders).
D. Criteria A, B and C must be confirmed by prospective daily ratings during at least two consecutive symptomatic cycles. (The diagnosis may be made provisionally prior to this confirmation).

The terms “treating” or “treatment” as used herein refer to:

(1) preventing or delaying the appearance of clinical symptoms of PMDD in a woman that may be afflicted with or predisposed to PMDD but does not yet experience or display clinical or subclinical symptoms of PMDD, or

(2) inhibiting PNFDD, i.e., arresting or reducing the development of PMDD or at least one clinical or subclinical symptom thereof, or

(3) reducing at least one clinical or subclinical symptom of PMDD.

The term “pharmaceutically acceptable” refers to additives or compositions that are physiologically tolerable and do not typically produce an allergic or similar untoward reaction, such as gastric upset, dizziness and the like, when administered to a mammal.

The term “effective amount” refers to an amount of escitalopram or a pharmaceutically acceptable salt thereof which, when administered to a female patient for PMDD, is sufficient to treat the same (e.g., an amount sufficient to alleviate the symptoms (e.g., psychological symptoms) of PMDD).

Pharmaceutically acceptable salts of escitalopram include, but are not limited to, salts formed with organic and inorganic acids. Examples of such organic salts are maleic, fumaric, benzoic, ascorbic, pamoic, succinic, oxalic, salicylic, methanesulfonic, ethanedisulfonic, acetic, propionic, tartaric, citric, gluconic, lactic, malic, mandelic, cinnamic, citraconic, aspartic, stearic, palmitic, itaconic, glycolic, p-amino-benzoic, glutamic, benzene sulfonic and theophylline acetic acid, as well as the 8-halotheophyllines, for example 8-bromotheophylline. Examples of such inorganic salts are hydrochloric, hydrobromic, sulfuric, sulfamic, phosphoric and nitric acids. Preferred pharmaceutically acceptable salts of escitalopram include, but are not limited to, escitalopram oxalate and escitalopram hydrobromide.

The oxalate of escitalopram may be prepared as described in U.S. Pat. No. Re. 34,712 and the base and other pharmaceutically acceptable salts may be obtained therefrom by standard procedures.

Methods for the preparation of solid pharmaceutical preparations are well known in the art. Tablets may thus be prepared by mixing the active ingredients with ordinary adjuvants and/or diluents and subsequently compressing the mixture in a convenient tabletting machine. Examples of adjuvants or diluents comprise: corn starch, lactose, talcum, magnesium stearate, gelatin, lactose, gums, and the like. Any other adjuvant or additive such as colorings, flavorings, preservatives, etc. may also be used provided that they are compatible with the active ingredients.

According to the invention, escitalopram or a pharmaceutically acceptable salt thereof may be administered in any suitable way e.g. orally or parenterally, and it may be presented in any suitable form for such administration, e.g. in the form of tablets, capsules, powders, syrups, or solutions or dispersions for injection. Preferably, and in accordance with the purpose of the present invention, the compound of the invention is administered in the form of a solid pharmaceutical entity, suitably as a tablet or a capsule or in the form of a suspension, solution, or dispersion for injection.

The pharmaceutical composition prepared according to the invention may comprise escitalopram in a daily dosage form containing 5-20 mg escitalopram, preferably 10-20 mg escitalopram, including 10 mg, 15 mg, and 20 mg, and most preferred 20 mg escitalopram.

“Menstrual cycle” refers to the reproductive cycle of female humans. The cycle is characterized by a monthly discharge of blood, mucus, and tissues from the uterus (called menstruation) and involves changes to the lining of the uterus (the endometrium) during the rest of the month including a few days of fertility after an ovum (egg) is released by an ovary.

“Luteal phase” refers to the segment of a patient's menstrual cycle beginning on the expected day of ovulation, as calculated individually from normal cycle length minus 14 days, and ending on the first day of full bleeding.

“Follicular phase” refers to the segment of a patient's menstrual beginning on the first day of full bleeding and ending the day before the expected day of ovulation, as calculated individually from normal cycle length.

“Continuously” refers in regards to dosage refers to administration of escitalopram or pharmaceutically acceptable salt thereof during every day of the menstrual cycle (i.e., throughout the menstrual cycle).

“Intermittently” in regards to dosage refers to administration of escitalopram or a pharmaceutically acceptable salt thereof for a number of sequential days that do not equal the total number of days in the patient's menstrual cycle. For example, a preferred intermittent dosage regiment is administration of the escitalopran or salt thereof during the luteal phase. A preferred regimen is the administration of escitalopram or salt thereof beginning at the expected day of ovulation as calculated individually from normal cycle length minus 14 days, and administering the last dose on the first day of full bleeding of the subsequent cycle. Examples of such a regimen are as follows:

(1) 5 mg escitalopram or a salt thereof is administered daily for the first 2 days, and 10 mg is administered thereafter until the first day of full bleeding;

(2) 5 mg escitalopram or a salt thereof may be administered daily for the first 1 day, 10 mg for 1 day (the second day), and 20 mg may be administered thereafter until the first day of full bleeding;

(3) 5 mg escitalopram or a salt thereof may be administered daily for the first 1 day, and 10 mg may be administered thereafter until the first day of full bleeding;

(4) 5 mg escitalopram or a salt thereof may be administered daily for the first 2 days, and a flexible daily dose between 5 mg and 20 mg can be self-administered by the patient thereafter until the first day of full bleeding; and

(5) depending on the severity of the symptoms, a flexible daily dose between 5 mg and 20 mg can be self-administered by the patient until the first day of full bleeding. As the length of the luteal phase will vary depending on the regularity and length of the individual patient's menstrual cycle, the number of days comprising the defined period of “intermittent dosage” may vary accordingly. Thus, daily dosage for escitalopram administration in any of the aforementioned examples can be adjusted to correlate with the patient's cycle.

“Semi-intermittently” in regards to dosage refers to administration of escitalopram or pharmaceutically acceptable salt thereof at a high dosage for a number of sequential days that corresponding to the patient's luteal phase, then administering a lower baseline dosage the days of the follicular phase of the patient's cycle. This may be a preferred embodiment in the case of a patient who exhibits symptoms of PMDD superimposed on other depressive disorders. Examples of such a regimen are as follows:

(1) 5 mg of escitalopram or salt thereof is administered daily during the follicular phase of the menstrual cycle, and 10 mg of escitalopram or salt thereof is administered beginning at the expected day of ovulation as calculated individually from normal cycle length minus 14 days, until the last dose on the first day of full bleeding of the subsequent cycle;

(2) 10 mg of escitalopram or salt thereof is administered daily during the follicular phase of the menstrual cycle, and 20 mg beginning at the expected day of ovulation until the last dose on the first day of full bleeding of the subsequent cycle;

(3) A low dosage from about 5 mg to about 10 mg of escitalopram or salt thereof is administered daily during the follicular phase of the menstrual cycle, then a higher dosage from about 10 mg to about 20 mg is administered beginning on the expected day of ovulation until the last dose on the first day of full bleeding of the subsequent cycle; and

(4) A constant or variable dosage of escitalopram or salt thereof is administered daily during the follicular stage, then a dosage of escitalopram or salt thereof, which is higher than any dosage administered during the follicular phase, is administered during the luteal phase.

As the length of the luteal phase will vary depending on the regularity and length of the patient's menstrual cycle, the number of days comprising the defined periods of luteal and follicular phases may vary accordingly. Thus, the daily dosage for escitalopram administration in any of the aforementioned examples of “semi-intermittent dosage” can be adjusted to correlate with the patient's cycle.

### EXAMPLES

The following examples illustrate the invention without limitation. All parts and percentages are given by weight unless otherwise indicated.

### Example 1

A double-blind, randomized, placebo-controlled trial evaluating the efficacy and safety of 3 months of intermittent treatment with 10 mg and 20 mg escitalopram per day in patients with PMDD is performed.

Intermittent treatment with 10 mg and 20 mg escitalopram oxalate daily is compared with placebo in a single-site, randomized, double-blind, parallel-group design.

150 patients are required for the Full Analysis Set. Patients eligible for this trial are female outpatients aged at least 18 years with a regular menstrual cycle who meet the DSM-IV-TR criteria for PMDD. The trial consists of two periods, the screening period of 2 menstrual cycles and the treatment period of 3 cycles. During the entire trial period patients are to complete a diary with daily rating of symptoms by means of VAS (Visual Analog Scales, ranging from 0-100 mm).

During treatment, escitalopram is administered in the luteal phase of the menstrual cycle. Patient contacts are to be undertaken a) in the luteal phase prior to the screening period for pre-screening, b) in the follicular phase of the cycle following the screening cycles for randomization, c) in the luteal phase of each treatment cycle and d) 28 days after the last trial visit for safety follow-up.

Escitalopram oxalate 10 mg and 20 mg daily (calculated by weight of escitalopram base) and placebo will be administered intermittently during the luteal phase of 3 menstrual cycles. Patients will start taking the escitalopram oxalate at the expected day of ovulation as calculated individually from normal cycle length minus 14 days. The last dose of study drug will be taken at the first day of full bleeding of the subsequent cycle. Patients should take the escitalopram oxalate in the morning.

The escitalopram dose will be uptitrated at each treatment cycle as follows:

(a) in the 10 mg group: 5 mg escitalopram oxalate daily for 2 days, then 10 mg escitalopram oxalate daily;

(b) in the 20 mg group: 5 mg escitalopram oxalate for 1 day, 10 mg escitalopram oxalate for 1 day, then 20 mg escitalopram oxalate daily

Escitalopram oxalate 5 mg, 10 mg and 20 mg tablets differ in size and shape. In order to achieve double-blinding of study drug, all escitalopram oxalate and placebo tablets will be encapsulated.

The treatment will consist of capsules, identical in appearance, taste and smell, which will contain escitalopram oxalate tablets with the strength 5 mg, 10 mg, 20 mg or placebo tablets.

A dose increase as a consequence of lack of efficacy is not planned, nor will the dose be down-regulated due to the appearance of adverse events. If the patient does not tolerate the dose administered, she will be withdrawn from the trial.

The following efficacy assessments will be carried out: 1) self-rated: VAS (Visual Analog Scales, ranging from 0-100 mm) of 10 premenstrual symptoms, SDS (Sheehan Disability Scale, a 3 item scale of impairment of functioning), PGE (Patient Global Evaluation, how ill the patient is feeling), and 2) observer-rated: CGI-S (Clinical Global Impression-Severity Scale), CGI-I (Clinical Global Impression-Improvement Scale) and PMTS-O (Premenstrual Tension Syndrome Scale-Observer Rating). The primary efficacy variable will be percent change from baseline (that is, average of 2 screening cycles) of mean of VAS of 4 key symptoms at treatment cycle 3. For the safety evaluation, vital signs (including weight) will be recorded and blood analyses carried out. Other assessments involve physical examination (including height), MINI (Mini-International Neuropsychiatric Interview, to screen for psychiatric disorders), MADRS (Montgomery-Asberg Depression Rating Scale, for depression), and self-assessment of sexual function parameters.

### Example 2

The procedure described in example 1 is repeated except the escitalopram oxalate is administered continuously throughout the menstrual cycle. The daily dose is 5, 10, 15, or 20 mg of escitalopram oxalate (calculated based on the weight of escitalopram base).

### Example 3

The procedure described in example 1 is repeated except the escitalopram oxalate is administered “semi-intermittently”, i.e., at a constant low dose during the follicular phase and a higher dose during the luteal phase. The regimen is:

(1) 5 mg escitalopram or a salt thereof is administered daily for the first 2 days, and 10 mg is administered thereafter until the first day of full bleeding;

(2) 5 mg escitalopram or a salt thereof may be administered daily for the first 1 day, 10 mg for 1 day (the second day), and 20 mg may be administered thereafter until the first day of fill bleeding;

(3) 5 mg escitalopram or a salt thereof may be administered daily for the first 1 day, and 10 mg may be administered thereafter until the first day of full bleeding;

(4) 5 mg escitalopram or a salt thereof may be administered daily for the first 2 days, and a flexible daily dose between 5 mg and 20 mg can be self-administered by the patient thereafter until the first day of full bleeding; or

(5) depending on the severity of the symptoms, a flexible daily dose between 5 mg and 20 mg can be self-administered by the patient until the first day of full bleeding.

All references cited herein are incorporated by reference. To the extent that a conflict may exist between the specification and the reference the language of the disclosure made herein controls.

1. A method of treating a woman suffering from premenstrual dysphoric disorder comprising administering an effective amount of escitalopram or a pharmaceutically acceptable salt thereof.

2. The method of claim 1, wherein the pharmaceutically acceptable salt of escitalopram is escitalopram oxalate.

3. The method of claim 1, wherein the pharmaceutically acceptable salt of escitalopram is escitalopram hydrobromide

4. The method of claim 1, wherein from about 5 to about 20 mg of escitalopram or a pharmaceutically acceptable salt thereof calculated on a weight basis of escitalopram base are administered daily.

5. The method of claim 4, wherein from about 5 to about 10 mg of escitalopram or a pharmaceutically acceptable salt thereof calculated on a weight basis of escitalopram base are administered daily.

6. The method of claim 4, wherein from about 10 to about 20 mg of escitalopram or a pharmaceutically acceptable salt thereof calculated on a weight basis of escitalopram base are administered daily.

7. The method of claim 4, wherein about 5 mg of escitalopram or a pharmaceutically acceptable salt thereof calculated on a weight basis of escitalopram base are administered daily.

8. The method of claim 4, wherein about 10 mg of escitalopram or a pharmaceutically acceptable salt thereof calculated on a weight basis of escitalopram base are administered daily.

9. The method of claim 4, wherein about 20 mg of escitalopram or a pharmaceutically acceptable salt thereof calculated on a weight basis of escitalopram base are administered daily.

10. The method of claim 1 wherein the escitalopram or a pharmaceutically acceptable salt thereof is administered orally.

11. The method of claim 10, wherein a solid dosage form of the escitalopram or a pharmaceutically acceptable salt thereof is administered.

12. The method of claim 11, wherein the solid dosage form is a tablet or capsule.

13. The method of claim 11, wherein the solid dosage form is administered once daily.

14. The method of claim 11, wherein the solid dosage form comprises from about 5 to about 20 mg of escitalopram or a pharmaceutically acceptable salt thereof calculated on a weight basis of escitalopram base.

15. The method of claim 14, wherein the solid dosage form comprises from about 5 to about 10 mg of escitalopram or a pharmaceutically acceptable salt thereof calculated on a weight basis of escitalopram base.

16. The method of claim 14, wherein the solid dosage form comprises from about 10 to about 20 mg of escitalopram or a pharmaceutically acceptable salt thereof calculated on a weight basis of escitalopram base.

17. The method of claim 14, wherein the solid dosage form comprises about 5 mg of escitalopram or a pharmaceutically acceptable salt thereof calculated on a weight basis of escitalopram base.

18. The method of claim 14, wherein the solid dosage form comprises about 10 mg of escitalopram or a pharmaceutically acceptable salt thereof calculated on a weight basis of escitalopram base.

19. The method of claim 14, wherein the solid dosage form comprises about 20 mg of escitalopram or a pharmaceutically acceptable salt thereof calculated on a weight basis of escitalopram base.

20. The method of claim 1, wherein the escitalopram is administered continuously throughout the menstrual cycle.

21. The method of claim 1, wherein the escitalopram is administered intermittently throughout the menstrual cycle.

22. The method of claim 1, wherein the escitalopram is administered semi-intermittently throughout the menstrual cycle.
